(19) United States
(12) Patent Application Publication
(10) Pub. No.: US 2022/0363956 A1
(43) Pub. Date: Nov. 17, 2022

(54) ADHESIVE SHEET, DEVICE LAMINATE, AND METHOD FOR PEELING ADHESIVE SHEET

(71) Applicant: 3M INNOVATIVE PROPERTIES COMPANY, St. Paul, MN (US)

(72) Inventors: Yasuhiro KINOSHITA, Tokyo (JP); Ryoko Kunibu, Saitama (JP)

(21) Appl. No.: 17/770,860

(22) PCT Filed: Nov. 23, 2020
(86) PCT No.: PCT/IB2020/061013

§ 371 Date: Apr. 21, 2022

(30) Foreign Application Priority Data
Nov. 25, 2019 (JP) 2019-212434

(51) Int. Cl. C09J 7/38; C09J 7/25; C09J 133/08

(52) U.S. Cl. CPC C09J 7/385; C09J 7/255; C09J 133/08; C09J 2301/122; C09J 2301/20; C09J 2301/302

(57) ABSTRACT

An adhesive sheet includes an adhesive layer including a surface provided with a fine structure. The fine structure includes a plurality of convex structures. Each of the convex structures includes two or more parts joined to each other via an interface. The two or more parts include a first part present at a top of the convex structure and made from an adhesive material, and a second part present in a lower side of the first part. The second part is harder than the first part. The adhesive sheet provides a gap between adjacent ones of convex structures of the adhesive sheet after the convex structures are applied to a subject.

### TECHNICAL FIELD

The present disclosure relates to an adhesive sheet, a device laminate, and a method for peeling an adhesive sheet.

### BACKGROUND

Breathable adhesive tape can including an adhesive layer formed in one surface of a backing made from nonwoven fabric is known (Patent Document 1: JP 3233732 B). The adhesive tape is applied to skin. Water vapor generated from the skin is released to the outside through a gap in the nonwoven fabric. That is, the water vapor moves in a direction orthogonal to the adhesive tape.

An adhesive sheet including an adhesive layer patterned in a corrugated form in a surface of a backing is also known (Patent Document 2: JP 3863634 B). The adhesive sheet is applied to skin. Water vapor generated from the skin is released to the outside through a corrugated space formed between the adhesive layers. That is, the water vapor moves in a direction parallel to the adhesive sheet.

### SUMMARY

In a case where the water vapor moves in the direction orthogonal to the adhesive tape as in the case of Patent Document 1, adequate breathability cannot be ensured when a non-breathable object is disposed on the adhesive tape. On the other hand, in a case where the water vapor is released to the outside through the corrugated space formed between the adhesive layers as in the case of Patent Document 2, there is a risk of crushing the adhesive layers and narrowing the corrugated space when the adhesive sheet is applied to skin.

The present disclosure provides an adhesive sheet enabling a gap formed between adjacent ones of convex structures of the adhesive sheet to be large after the convex structures are applied to a subject. Further, the present disclosure provides an adhesive sheet including such an adhesive sheet and a method for peeling such an adhesive sheet.

The disclosed adhesive sheet enables a gap formed between adjacent ones of convex structures of the adhesive sheet to be large after the convex structures are applied to a subject. Further, an adhesive sheet including such an adhesive sheet and a method for peeling such an adhesive sheet can be provided.

In one embodiment, the adhesive sheet includes a surface provided with a fine structure. The fine structure includes a plurality of convex structures. Each of the plurality of convex structures includes two or more parts joined to each other via an interface. The two or more parts include a first part present at a top of the convex structure and made from an adhesive material, and a second part present in a lower side of the first part. The second part is harder than the first part.

The second part may have a storage elastic modulus higher than a storage elastic modulus of the first part.

A storage elastic modulus of the second part may be 1×10⁵ Pa or more.

A storage elastic modulus of the first part may be less than 1×10⁵ Pa.

An angle θ formed between a side surface and a bottom surface of the convex structure may be 30° or more.

When a height of the convex structure is 100%, a height of the first part may be in the range of 10% to 90% of the height of the convex structure.

An interval between bottom surfaces of adjacent ones of the plurality of convex structures in an array direction of the plurality of convex structures may be 500 μm or less.

A device laminate according to an aspect of the present disclosure includes the adhesive sheet of the present disclosure, a device, a substrate disposed between the adhesive sheet and the device, and a connection layer disposed between the substrate and the device. The adhesive layer of the adhesive sheet includes a surface close to the substrate and a surface far from the substrate, and the adhesive layer includes the fine structure in the surface far from the substrate.

A method for peeling an adhesive sheet according to an aspect of the present disclosure includes a step of peeling the adhesive sheet from a subject in a state where the plurality of convex structures of the adhesive sheet are applied to the subject, by making a stripping solution flow in a gap formed between adjacent ones of the plurality of convex structures.

### BRIEF DESCRIPTION OF THE DRAWINGS

FIG. 1 is a perspective view of an adhesive sheet according to an embodiment.

FIG. 2 is a cross-sectional view taken along line II-II of FIG. 1.

FIG. 3 is a cross-sectional view of an adhesive sheet according to another embodiment.

FIG. 4 is a cross-sectional view of an adhesive sheet according to another embodiment.

FIG. 5 is a partial cross-sectional view of an adhesive sheet according to another embodiment.

FIG. 6 is a cross-sectional view illustrating other examples of a cone structure.

FIG. 7 is a cross-sectional view illustrating other examples of a frustum structure.

FIG. 8 is a perspective view of an adhesive sheet according to another embodiment.

FIG. 9 is a cross-sectional view illustrating a step in a method for manufacturing the adhesive sheet of FIG. 3.

FIG. 10 is a cross-sectional view illustrating a step following the step of FIG. 9.

FIG. 11 is a cross-sectional view illustrating a step following the step of FIG. 10.

FIG. 12 is a cross-sectional view illustrating a step of applying the adhesive sheet of FIG. 3 to a subject.

FIG. 13 is a cross-sectional view illustrating an adhesive sheet attached to another subject.

FIG. 14 is a cross-sectional view of a device laminate according to an embodiment.

FIG. 15 is a cross-sectional view illustrating a step in a method for peeling an adhesive sheet according to an embodiment.

FIG. 16 is a graph showing 180° peel adhesion strength of adhesive sheets of examples and reference examples.

FIG. 17 is a drawing illustrating the adhesive sheets before and after application in the examples and the reference examples.

### DETAILED DESCRIPTION

Detailed description of embodiments are given below with reference to the attached drawings. In the description of the drawings, the same or equivalent elements are denoted by the same reference signs, and redundant description of such elements will be omitted. The XYZ rectangular coordinate system is illustrated in the drawings as necessary.

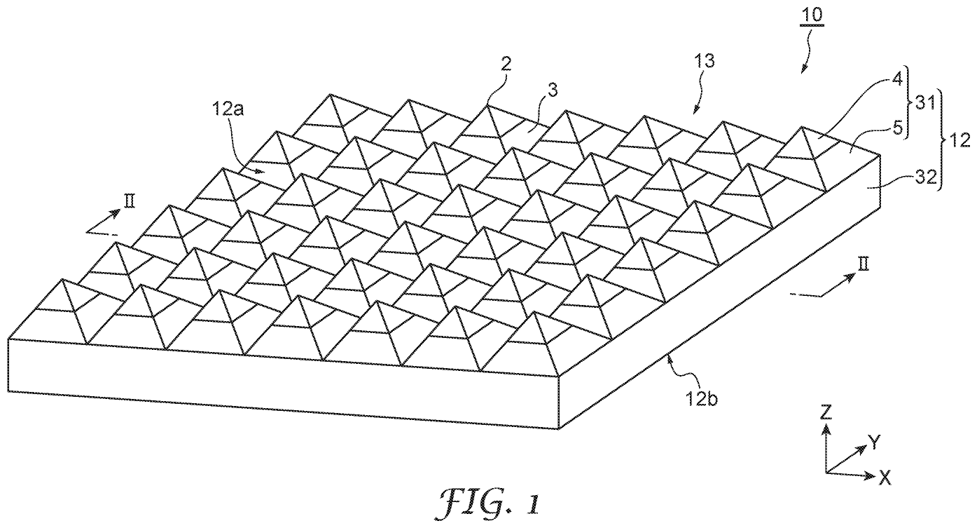

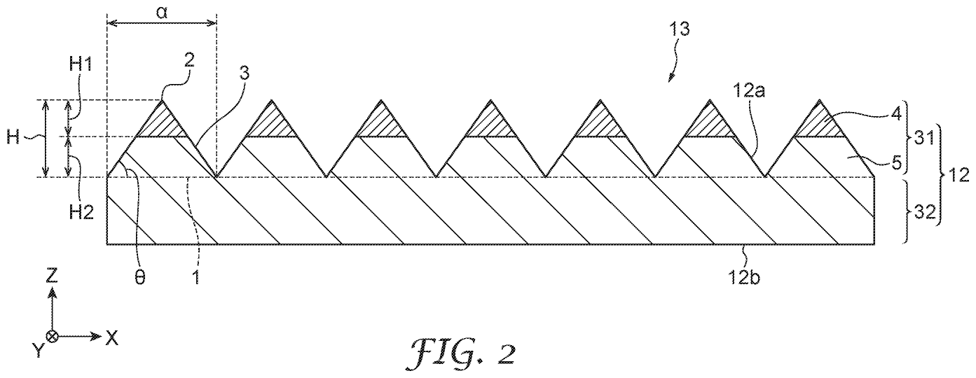

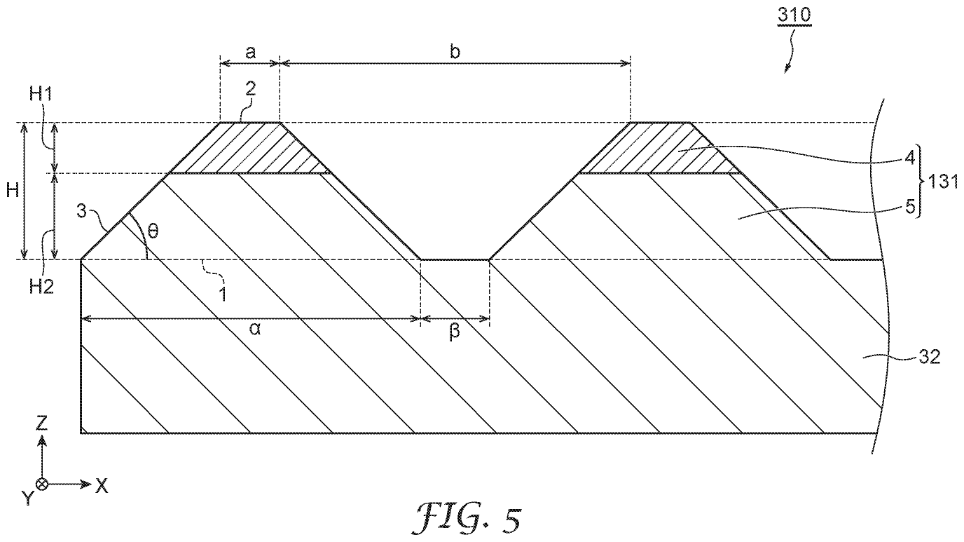

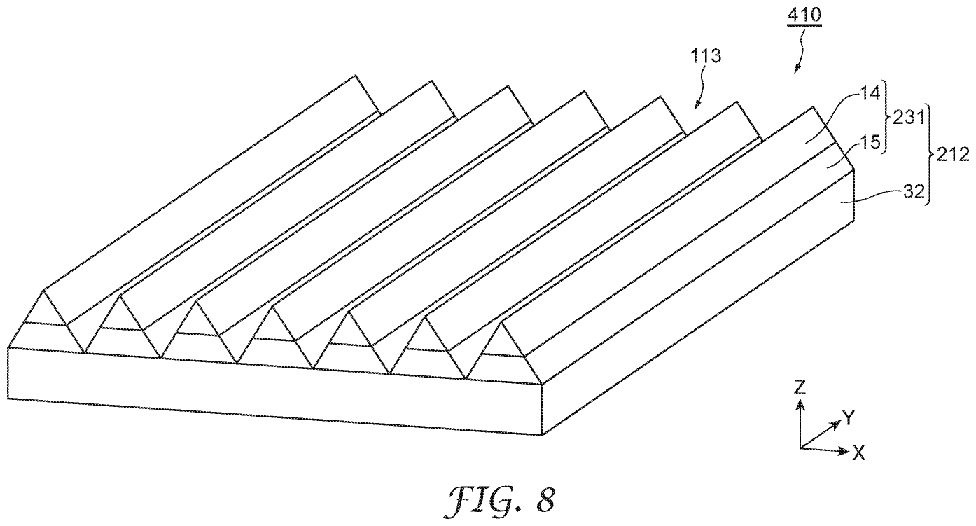

FIG. 1 is a perspective view of an adhesive sheet according to an embodiment. FIG. 2 is a cross-sectional view taken along line II-II of FIG. 1. An adhesive sheet 10 illustrated in FIGS. 1 and 2 includes an adhesive layer 12. In the present embodiment, the adhesive layer 12 includes one surface 12a provided with a fine structure 13 and the other surface 12b provided with no fine structure. The surface 12a and the surface 12b extend along a plane (e.g., an XY plane) orthogonal to a thickness direction (e.g., Z-axis direction) of the adhesive layer 12. The fine structure 13 includes a plurality of cone structures 31. The cone structure 31 may be replaced with a frustum structure 131 (FIG. 5) or a rib structure 231 (FIG. 8) described below. The cone structure 31, the frustum structure 131, and the rib structure 231 are each an example of a convex structure (convex body). Herein, the “convex structure” generally refers to a solid figure that includes any plane figure as a bottom surface, and that is constructed by connecting all points in a side of the bottom surface and all points in a side of any other plane figure or a line (top) that is not on the bottom surface. Preferably, the area of the top of the convex structure is smaller than the area of the bottom surface. More preferably, the convex structure is shaped to be tapered from the bottom surface toward the top. The plurality of cone structures 31 are arrayed along the X-axis direction and the Y-axis direction to form a lattice shape in the surface 12a. FIG. 2 is a cross-sectional view through apices of the plurality of cone structures 31 arrayed along the X-axis direction. The plurality of cone structures 31 can preferably be regularly arrayed or randomly arrayed on a plane. The area of each of the cone structures 31 projected to the plane orthogonal to the height direction of the cone structure 31 (area of a bottom surface 1 of the cone structure 31) may be 10 square micrometers or more and may be 10000 square micrometers or less.

Each cone structure 31 has the bottom surface 1, a top 2, and a plurality of side surfaces 3 connecting edges of the bottom surface 1 and the top 2. The bottom surface 1 has any plane figure such as a circle (including an ellipse) or a polygon. Examples of a shape of the cone structure 31 include a cone, a triangular pyramid, a quadrangular pyramid, and a hexagonal pyramid. In the example illustrated in FIGS. 1 and 2, the shape of the cone structure 31 is a quadrangular pyramid. The cone structures 31 may have the same shape or different shapes, but preferably have substantially the same height (with a difference within ±5%, ±3%, or ±1%) and more preferably all have substantially the same shape. In a case where the cone structures 31 have different shapes, the fine structure 13 preferably includes 10 or less, 9 or less, 8 or less, 7 or less, 6 or less, 5 or less, 4 or less, 3 or less, or 2 or less types of cone structures 31. Two or more of the cone structure 31, the frustum structure 131 (FIG. 5) and the rib structure 231 (FIG. 8) may coexist.

Each cone structure 31 includes a first part 4 present at the top 2 of the cone structure 31 and a second part 5 present in a lower side (the bottom surface 1 side) of the first part 4.

The top 2 is a part substantially occupying a region located at the highest position of the cone structure 31 (a part of the cone structure 31 that initially comes into contact with a subject when the adhesive sheet of the present disclosure approaches the subject) The top 2 preferably includes the apex of the cone structure 31. “Substantially occupying” means that the case where a different material is attached to or incorporated in only a part is also acceptable. For example, the first part 4 may occupy a majority (e.g., 90% or more, or 95% or more) of the region located at the highest position of the cone structure 31. Even when a small amount of a filler or the like is incorporated in the region, the filler or the like does not correspond to the first part 4.

The first part 4 and the second part 5 can be joined to each other via an interface along an XY plane for example. The being “joined via an interface” means a state where two matrix phases having different kinds of composition are in contact via a distinct interface. For example, the first part 4 (matrix phase) and the second part 5 (matrix phase) are layered and separated as illustrated in FIGS. 1 and 2 and thus are joined via the interface. Note that for example, in the case of a composition in which fine particles are dispersed in a resin, the resin serving as a substrate corresponds to the matrix phase, while the fine particles correspond to a dispersed phase. The being joined via an interface does not include joining of two phases including a common matrix phase and different dispersed phases, or a joining manner in which a material varies continuously, for example, in a material in which fine particles are dispersed in a resin, only density of the fine particles continuously varies in a direction. The interface may be a plane parallel or not parallel to the bottom surface 1 of the cone structure 31. The interface may include a surface curved due to, for example, a manufacturing error or surface tension in a manufacturing method described below. The cone structure 31 may optionally further include a third part, or may have a multilayer structure including three or more layers.

The first part 4 is made from an adhesive material. A known material used in manufacturing of a pressure sensitive adhesive can be used as the adhesive material. Among those, a material that can exhibit adhesive force to a subject while being repeelable is preferable. In an embodiment, the adhesive material can be defined as a material that meets a condition where a storage elastic modulus (G′) obtained by measuring at 37° C. or lower and a frequency of 1 Hz is less than about 1×10⁵ Pa. Thus, in an embodiment, a storage elastic modulus (G′) at 37° C. of the first part 4 is less than 1×10⁵ Pa. Specific examples of the adhesive material include an acrylic adhesive, a rubber-based adhesive, or a silicone-based adhesive. In the adhesive material, a tackifier, a plasticizer, and the like may be blended.

The second part 5 is harder than the first part 4. Hardness can be measured, for example, by using an atomic force microscope (AFM). The second part 5 is made from, for example, a non-adhesive or weak adhesive material. The weak adhesive material may be a material having adhesiveness lower than adhesiveness of the adhesive material of the first part 4. As the non-adhesive or weak adhesive material, a material having no adhesiveness to a subject, or a material having adhesiveness but being repeelable from a subject is preferable. The non-adhesive or weak adhesive material may be a so-called elastomer. In an embodiment, the non-adhesive or weak adhesive material is a resin having a storage elastic modulus (G′) calculated by dynamic viscoelasticity measurement of 1×10⁵ Pa or more, 3×10⁵ Pa or more, 4×10⁵ Pa or more, 5×10⁵ Pa or more, 6×10⁵ Pa or more, 7×10⁵ Pa or more, 8×10⁵ Pa or more, 9×10⁵ Pa or more, or 1×10⁶ Pa or more as measured at 37° C. or lower and a frequency of 1 Hz. Thus, in an embodiment, a storage elastic modulus (G′) at 37° C. of the second part 5 is higher than the storage elastic modulus (G′) of the first part 4, and is 1×10⁵ Pa or more. Specific examples of the non-adhesive or weak adhesive material include polyurethane, poly (meth) acrylate, cellulose, silicone, an amine-based resin, a fluorine-based resin, a synthetic rubber, and polyvinyl chloride. The non-adhesive or weak adhesive material preferably has high solubility and/or dispersibility in any general purpose solvent of a water miscible solvent such as water and alcohol, or a water immiscible solvent such as hydrocarbon. Additionally, a solvent in which the non-adhesive or weak adhesive material dissolves and/or disperses preferably has a relatively low vapor pressure and is easy to dry. Further, wettability to a mold for forming the fine structure 13 is preferably also considered. When the wettability is too low, the solvent may not enter an inside of a recess of the mold, and when the wettability is too high, the solvent may remain inside the recess of the mold.

Note that “non-adhesive,” “weak adhesive,” and “adhesive” mean relative strength of adhesiveness to the same subject. Adhesiveness can be evaluated by a known technique such as dynamic viscoelasticity measurement or a 180° peeling strength test.

A combination of the material of the first part 4 and the material of the second part 5 is not limited, but the materials are more preferably selected in consideration of adhesive force between the first part 4 and the second part 5. For example, from a perspective of affinity of a polymer structure and the like, when the material of the first part 4 is silicone, the material of the second part 5 is also preferably a silicone-based adhesive. However, the first part 4 and the second part 5 are not necessarily polymers having the same structure.

The adhesive layer 12 may include a base 32 below the plurality of cone structures 31. The base 32 is joined or continuous with the bottom surfaces 1 of the cone structures 31 of the fine structure 13. A material of the base 32 may be the same as or different from the material of the second part 5. In an embodiment, the cone structure 31 includes two parts that are the first part 4 and the second part 5, and the base 32 is made from the same material as the material of the second part 5, and continuous with the second part 5. The thickness of the base 32 can arbitrarily be set according to a desired thickness of the adhesive layer 12. When any of the adhesive material constituting the first part 4, the non-adhesive or weak adhesive material constituting the second part 5, and, when present, materials constituting other parts is transparent, the adhesive layer 12 can entirely be made transparent. At that time, to make the interface via which the parts are joined invisible, a difference in a refractive index among the materials constituting these parts is preferably within 1%. Specifically, when the first part 4 and the second part 5 of the cone structure 31 are adjacent to each other and the difference between the refractive index of the material constituting the first part 4 and the refractive index of the material constituting the second part 5 is within 1%, within 0.9%, within 0.8%, within 0.7%, or within 0.6%, the interface between the two parts is generally invisible. For example, when the first part 4 includes a transparent acrylic adhesive and the second part 5 includes a transparent acrylic resin, the above-described requirement is satisfied, and the adhesive layer 12 completely transparent can be provided. Note that transparent can be defined by, for example, haze of 40% or less as measured in accordance with JIS K 7136.

From a perspective of, for example, facilitating formation of the first part 4, the longest distance between the centers of two cone structures 31 adjacent to each other in the fine structure 13 may be 2 mm or less, 1 mm or less, 500 μm or less, 300 μm or less, or 100 μm or less. Note that the center of the cone structure 31 means an apex of a cone. The center of the frustum structure 131 (FIG. 5) means an apex of a corresponding cone structure.

From a perspective of, for example, facilitating formation of the first part 4, the width (corresponding to α described below) of the bottom surface 1 of the cone structure 31 in an array direction (the X-axis direction, for example) of the cone structures 31 may be 2 mm or less, 1 mm or less, 500 μm or less, 300 μm or less, 100 μm or less, or 50 μm or less.

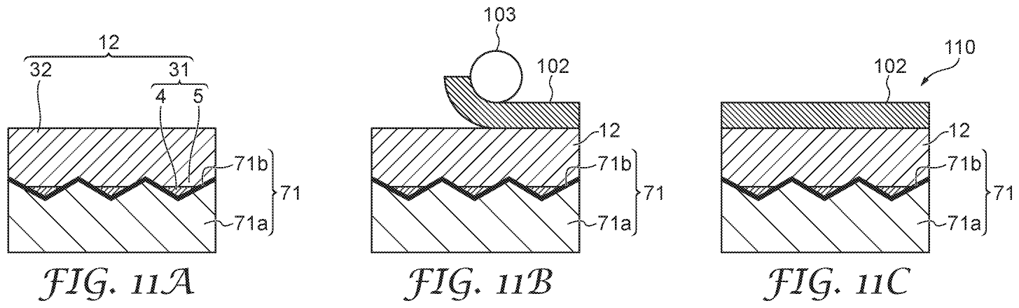

From a perspective of, for example, facilitating manufacturing of the adhesive sheet 10 or facilitating peeling of a liner 71 (see FIG. 11) from the adhesive sheet 10 completed, a height H of the cone structure 31 may be 5 μm or more, and 300 μm or less, 200 μm or less, 100 μm or less, 75 μm or less, or 50 μm or less.

From a perspective of providing sufficient adhesiveness, the number of the cone structures 31 is preferably 25 or more, 36 or more, 49 or more, 64 or more, 81 or more, or 100 or more per cm² of a surface of the adhesive layer 12. The number of the cone structures 31 corresponds to the number of the centers of the cone structures 31 present in the unit area. High density of the cone structures 31 also contributes to improvement of adhesiveness.

The bottom surfaces 1 of two cone structures 31 adjacent to each other may be close to each other. For example, in the case of a quadrangular pyramid or a hexagonal pyramid, the bottom surfaces 1 of two cone structures 31 adjacent to each other may share one side, or adjacent sides may be separate at an interval (corresponding to β described below) of, for example, 500 μm or less, 400 μm or less, 300 μm or less, 200 μm or less, 150 μm or less, 100 μm or less, 50 μm or less, 15 μm or less, or 10 μm or less.

Assuming that α represents the width of the bottom surface 1 of the cone structure 31 and β represents the interval between the bottom surfaces 1 of the cone structures 31 adjacent to each other in the array direction (the X-axis direction for example) of the cone structures 31, β/(α+β)<0.3, β/(α+β)<0.2, or β/(α+β)<0.1 may be satisfied. Thus, in the example illustrated in FIGS. 1 and 2, since β is 0, a value of β/(α+β) is 0. The plurality of cone structures 31 may be arrayed at an interval β greater than 0 between the bottom surfaces 1 of the cone structures 31 adjacent to each other.

Note that (α+β) corresponds to pitch of the cone structures 31 adjacent to each other. This (α+β) may be 10 μm or more, 20 μm or more, 30 μm or more, and 2.5 mm or less, 1 mm or less, 500 μm or less, 200 μm or less, 150 μm or less, or 100 μm or less.

From a perspective of, for example, facilitating formation of the first part 4 or facilitating formation of a gap between the cone structures 31 adjacent to each other, an angle θ formed between the side surface 3 and the bottom surface 1 of the cone structure 31 can be 5° or more, 10° or more, 15° or more, 20° or more, 25° or more, or 30° or more in a cross section (XZ plane) including the apices of the cone structures 31 and the array direction of the cone structures 31. Additionally, from a perspective of smoothly peeling the adhesive sheet 10 from the liner 71 described below, the angle θ may be less than 90°, 85° or less, 80° or less, or 70° or less in the cross section (XZ plane) including the apices of the cone structures 31 and the array direction of the cone structures 31.

The height H of the cone structure 31 may be 5 μm or more, 10 μm or more, or 30 μm or more, and 300 μm or less, 200 μm or less, or 100 μm or less. From a perspective of adhesiveness and the like, when the height H of the cone structure 31 is 100%, a height H1 of the first part 4 may be 10% or more, 15% or more, or 20% or more of the height H of the cone structure 31. Additionally, from a perspective of facilitating formation of a gap between the cone structures 31 adjacent to each other, the height H1 of the first part 4 may be 90% or less, 80% or less, 70% or less, 60% or less, or 50% or less of the height H of the cone structure 31. Note that the heights H and H1 are based on the normal direction (the Z-axis direction) of the bottom surface 1 of the cone structure 31. When the interface between the first part 4 and the second part 5 located below the first part 4 is a plane or a curved surface that is not parallel to the bottom surface 1, the height H1 is calculated from an average value of the heights of the interface determined based on the normal direction of the bottom surface 1. When the first part 4 is relatively small, adhesiveness of the adhesive sheet 10 tends to reduce. On the other hand, when the first part 4 is relatively large, the opposite is true.

The thickness of the adhesive layer 12 can arbitrarily be set according to the adhesive material used, the intended use of the adhesive sheet 10, or the like, and can be, for example, in the range of 15 μm to 1 mm or 50 μm to 300 μm. The thickness of the adhesive layer 12 means the distance between the highest part of the cone structure 31 and the surface 12b opposite to the surface 12a provided with the fine structure 13, based on the normal direction of the bottom surface 1 of the cone structure 31.

The adhesive layer 12 may include an additional material other than an adhesive, for example, fine particles such as hollow or solid glass spheres for the purpose of adjusting adhesiveness. However, the adhesive sheet 10 of the present disclosure can achieve desired properties without including such additional materials. In an embodiment, the adhesive layer 12 includes no fine particle.

### Characteristics of Adhesive Sheet

The adhesive sheet 10 exhibits sufficient adhesive force to a subject when relatively high pressure is applied to the surface 12b of the adhesive layer 12. In an embodiment, the “relatively high pressure” can be defined as pressure corresponding to pressure generated by reciprocating a roller of 2 kg at a speed of 300 mm/minute by using a compression bonding apparatus defined in 10.2.4 of JIS Z 0237: 2009. In another embodiment, the “relatively high pressure” can be defined as pressure of 200 g/cm² or more, 300 g/cm² or more, 400 g/cm² or more, 500 g/cm² or more, 600 g/cm² or more, or 700 g/cm² or more. In a preferred embodiment, the adhesive sheet 10 has 90° peel adhesion strength as tested for an SUS plate under conditions of a temperature of 23° C. and a tensile speed of 300 mm/minute of 0.2 N/10 mm or more, 1 N/10 mm or more, 2 N/10 mm or more, 4 N/10 mm or more, 6 N/10 mm or more, 8 N/10 mm or more, or 10 N/10 mm or more in 24 hours after adhesion. When such adhesive force is exerted, the adhesive sheet 10 is less likely to peel off or the like after adhesion of the adhesive sheet 10.

As described above, in the adhesive sheet 10, the second part 5 is harder than the first part 4, and thus the second part 5 is less likely to deform than the first part 4 when the cone structures 31 of the adhesive sheet 10 are applied to a subject. Thus, after the cone structures 31 of the adhesive sheet 10 are applied to a subject, a large gap can be formed between the cone structures 31 adjacent to each other. In an example, when the cone structures 31 of the adhesive sheet 10 are applied to skin, a gap is formed between the cone structures 31 adjacent to each other. Water vapor generated from the skin is released to the outside through the gap. That is, the gap functions as a flow path for the water vapor. Additionally, the first part 4 is relatively soft, and thus when the adhesive sheet 10 is applied to skin, impact on the skin is small, and when the adhesive sheet 10 is peeled from the skin, the adhesive sheet 10 is less likely to pull up the skin. In another example, when the cone structures 31 of the adhesive sheet 10 are applied to a wall, a gap is formed between the cone structures 31 adjacent to each other. The adhesive sheet 10 can be peeled from the wall by making a stripping solution flow in the gap. That is, the gap functions as a flow path for the stripping solution.

When the second part 5 has a higher storage elastic modulus than a storage elastic modulus of the first part 4, the second part 5 can be harder than the first part 4. The storage elastic modulus of the second part 5 is, for example, 1×10⁵ Pa or more. The storage elastic modulus of the first part 4 is, for example, less than 1×10⁵ Pa.

When an angle θ formed between the side surface 3 and the bottom surface 1 of the cone structure 31 is 30° or more, closing by the first part 4 of the gap between the cone structures 31 adjacent to each other can be suppressed when the cone structures 31 of the adhesive sheet 10 are applied to a subject.

When the height H of the cone structure 31 is 100%, the height H1 of the first part 4 may be in the range of 10% to 90% of the height H of the cone structure 31. When the cone structures 31 of the adhesive sheet 10 are applied to a subject, the volume of the gap formed between the cone structures 31 adjacent to each other can be adjusted by adjusting the height H of the cone structure 31.

When the interval 13 between the bottom surfaces 1 of the cone structures 31 adjacent to each other is 500 μm or less in the array direction of the cone structures 31, the plurality of cone structures 31 can be arrayed densely in the array direction of the cone structures 31. Thus, when the cone structures 31 of the adhesive sheet 10 are applied to a subject, adhesiveness of the adhesive sheet 10 to the subject can be improved.

Such an adhesive sheet 10 can be applied to various kinds of applications. A subject to which the adhesive sheet 10 is applied may be, for example, a superficial part of a human body or an animal such as skin, or may be an object such as a wall material, a floor material, a tile material, a sash material, a signboard, an electronic device, and an electronic part. The adhesive sheet 10 is, for example, a surgical tape (tape for a medical purpose).

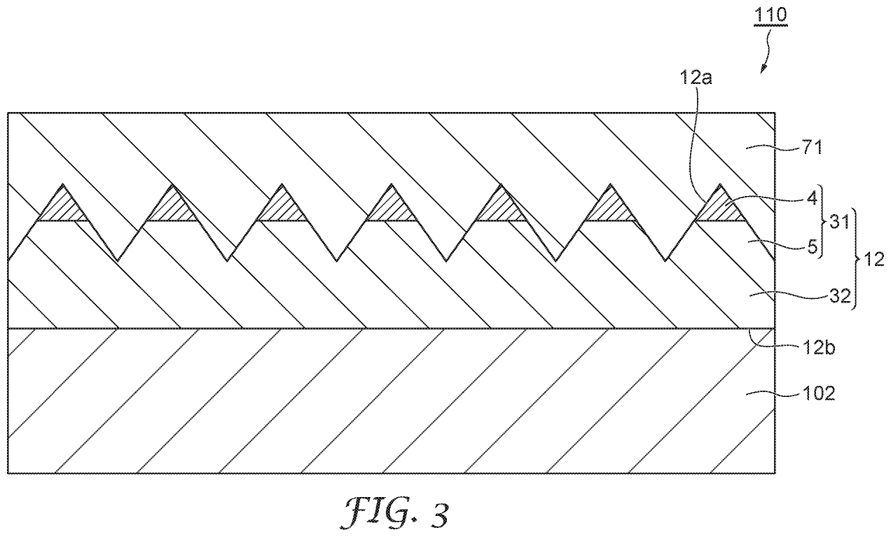

FIG. 3 is a cross-sectional view of an adhesive sheet according to another embodiment. An adhesive sheet 110 illustrated in FIG. 3 includes the adhesive layer 12 illustrated in FIGS. 1 and 2, a liner 71 disposed on a fine structure 13, and a carrier 102 provided in a surface 12b provided with no fine structure. The liner 71 can protect the fine structure 13. The adhesive sheet 110 may not include any one of the liner 71 and the carrier 102. For example, when the adhesive sheet 110 does not include the carrier 102, a roll can be formed by winding the adhesive sheet 110 around a core with the adhesive layer 12 on the inner side.

Examples of the carrier 102 include nonwoven fabric, woven fabric, a resin film, for example, a film made from ABS, ASA, acrylic, polycarbonate, polyurethane, fluororesin, polypropylene, PET, or PVC. The adhesive sheet 110 can include any layer including a primer or the like between the carrier 102 and the adhesive layer 12.

Examples of the liner 71 include a film made from a material similar to the material of the carrier 102.

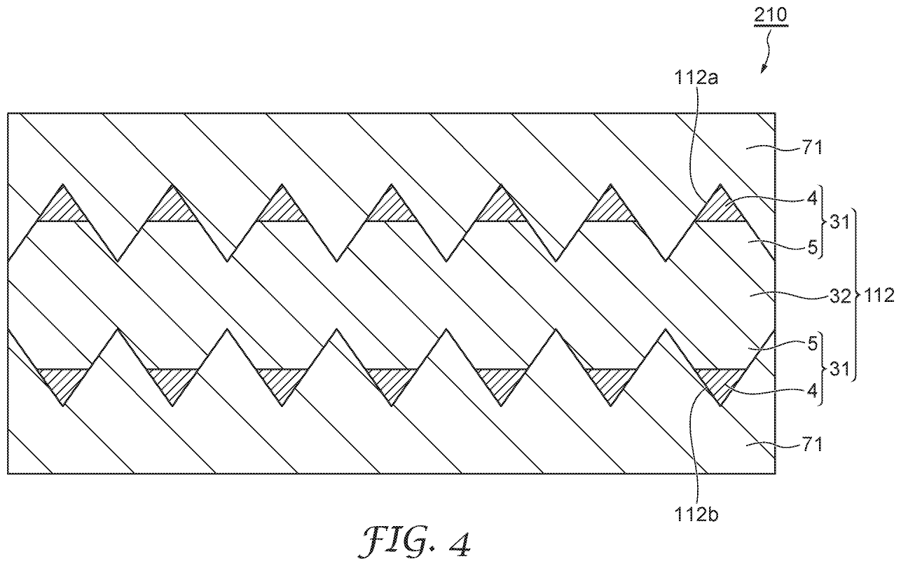

FIG. 4 is a cross-sectional view of an adhesive sheet according to another embodiment. An adhesive sheet 210 illustrated in FIG. 4 includes an adhesive layer 112 and a pair of liners 71 sandwiching the adhesive layer 112. The adhesive layer 112 includes a configuration in which a fine structure 13 is provided in the surface 12b of the adhesive layer 12 illustrated in FIGS. 1 and 2. Thus, both surfaces 112a and 112b of the adhesive layer 112 are each provided with the fine structure 13. The fine structures 13 provided in the surfaces 112a and 112b may have the same structure or may have different structures. For example, a material or a height H1 of a first part 4 can be the same or different between the surfaces 112a and 112b.

FIG. 5 is a partial cross-sectional view of an adhesive sheet according to another embodiment. An adhesive sheet 310 illustrated in FIG. 5 includes the same configuration as the configuration of the adhesive sheet 10 except that the adhesive sheet 310 includes a plurality of frustum structures 131 instead of the plurality of cone structures 31, and that the plurality of frustum structures 131 are disposed at an interval in the array direction. Each of the frustum structures 131 includes a structure obtained by partially cutting off an uppermost part of the cone structure 31 including the apex. Examples of a shape of the frustum structure 131 includes a truncated cone, a triangular truncated cone, a quadrangular truncated cone, a hexagonal truncated cone, and the like. Each frustum structure 131 includes a first part 4 present at a top 2 of the frustum structure 131 and a second part 5 present in a lower side (a bottom surface 1 side) of the first part 4.

In the present embodiment, β/(α+β)<0.3 is satisfied where a represents the width of the bottom surface 1 of the frustum structure 131 and β represents an interval between the bottom surfaces 1 of the frustum structures 131 adjacent to each other, in the array direction of the frustum structures 131 (X-axis direction, for example). In the example of FIG. 5, β is greater than 0, but β may be 0. In the array direction of the frustum structures 131, the width of a top surface of the frustum structure 131 is defined as a and an interval between the top surfaces of the frustum structures 131 adjacent to each other is defined as b. When a is 0, the frustum structure 131 includes the same structure as the cone structure 31.

The width a of the top surface of the frustum structure 131 in the array direction of the frustum structures 131 is, for example, 1.5 mm or less, 1 mm or less, 500 μm or less, 50 μm or less, 40 μm or less, 30 μm or less, 20 μm or less, or 10 μm or less. Reduction of adhesive force exerted under pressure of a certain level or more can be prevented by making the width a of the top surface not too large with respect to the width a of the bottom surface 1.

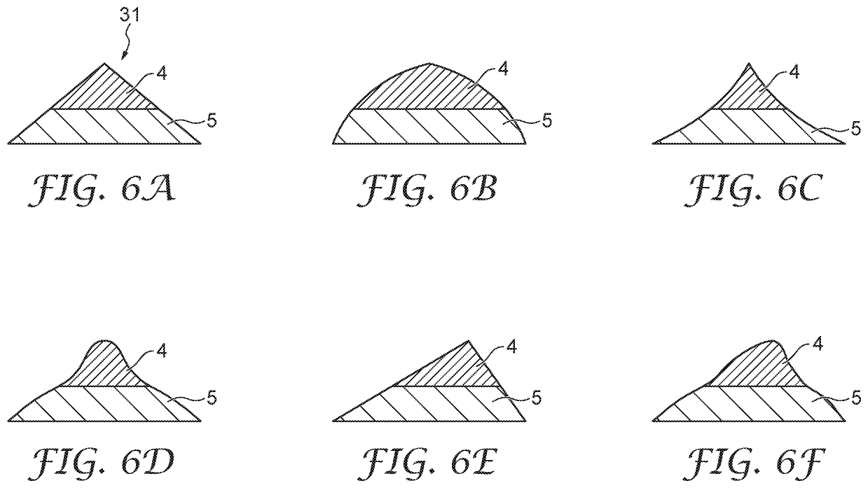

FIG. 6 is a cross-sectional view illustrating other examples of the cone structure. A cross section of the cone structure 31 can have a triangular shape as illustrated in (A) of FIG. 6, may have distorted side surfaces as illustrated in (B) to (D), or may have a shape in which a position of an apex is shifted from the center of a bottom surface as illustrated in (E). As illustrated in (F) of FIG. 6, a cross section of the cone structure 31 can have a distorted side surfaces and has a shape in which a position of an apex is shifted from the center of a bottom surface. Note that all the cross sections passing through the apices of the cone structures 31 do not necessarily have the same shape, and may have different shapes for each cross section.

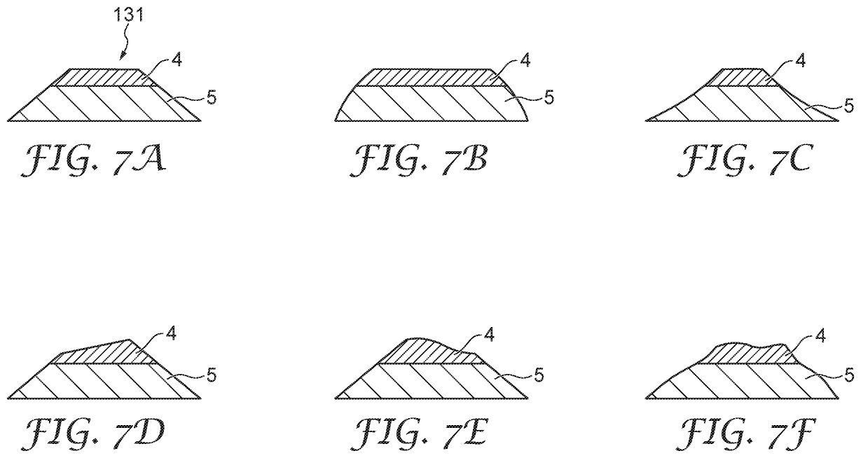

FIG. 7 is a cross-sectional view illustrating other examples of the frustum structure. A cross section of the frustum structure 131 may have a trapezoidal shape as illustrated in (A) of FIG. 7, may have distorted side surfaces as illustrated in (B) to (C), or may have a distorted top surface illustrated in (D) to (E). As illustrated in (F) of FIG. 7, a cross section of the frustum structure 131 may have distorted side surfaces and a distorted top surface. Note that all the cross sections passing through the apices of cones corresponding to the frustum structures 131 do not necessarily have the same shape, and may have different shapes for each cross section. Additionally, the top surface of the frustum structure 131 may not be in parallel with the bottom surface, or may not be a flat surface.

FIG. 8 is a perspective view of an adhesive sheet according to another embodiment. An adhesive sheet 410 illustrated in FIG. 8 includes the same configuration as the configuration of the adhesive sheet 10 illustrated in FIG. 1 except that the adhesive sheet 410 includes a plurality of rib structures 231 instead of the plurality of cone structures 31. The adhesive sheet 410 includes an adhesive layer 212 including a fine structure 113 including the plurality of rib structures 231. The plurality of rib structures 231 are arrayed along the X-axis direction, and each rib structure 231 extends in the Y-axis direction. Each rib structure 231 includes a first part 14 present at a top of the frustum structure 231 and a second part 15 present in a lower side (bottom surface side) of the first part 14. A cross section of the adhesive sheet 410 orthogonal to the Y-axis direction is the same as the cross section of the adhesive sheet 10 illustrated in FIG. 2. Materials and XZ cross-sectional shapes of the first part 14 and the second part 15 are the same as the materials and the XZ cross-sectional shapes of the first part 4 and the second part 5 of the adhesive sheet 10 illustrated in FIG. 2.

The rib structure 231 is a solid figure that includes, as a bottom surface, a plane figure in which a length in any axial direction (Y-axis direction) on a plane is greater than a length in an axial direction (X-axis direction) orthogonal to the axis, and that is constituted by connecting all points in a side of the bottom surface and all points in a line or a side of a rectangle extending in a direction substantially parallel to the Y-axis direction. A cross section of the rib structure 231 can have any shape as illustrated in FIGS. 6(A) to 6(F) and FIGS. 7(A) to 7(F) as with the cone structure 31 and the frustum structure 131. A ratio of the length in the Y-axis direction of the bottom surface of the rib structure 231 to the length in the X-axis direction, that is, an aspect ratio is, for example, 2 or more, 3 or more, 4 or more, 5 or more, 10 or more, 50 or more, 100 or more, 500 or more, 1000 or more, or 10000 or more. The rib structure 231 may be formed continuously along any axial direction across the entire surface of the adhesive sheet 410.

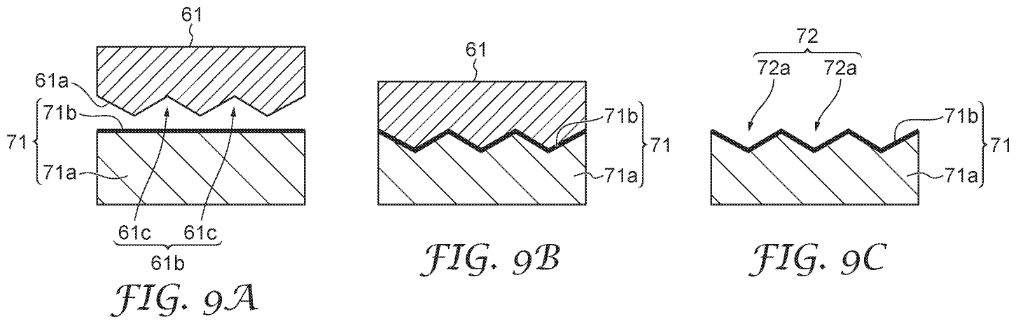

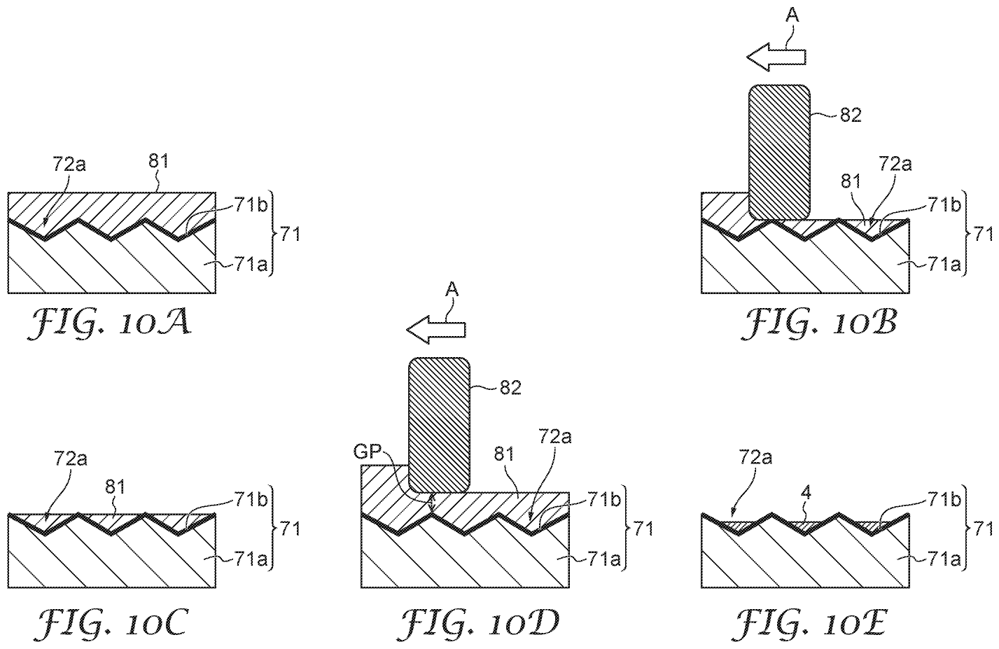

FIG. 9 is a cross-sectional view illustrating a step in a method of manufacturing the adhesive sheet of FIG. 3. FIG. 10 is a cross-sectional view illustrating a step following the step of FIG. 9. FIG. 11 is a cross-sectional view illustrating a step following the step of FIG. 10. The adhesive sheet 110 of FIG. 3 can be manufactured, for example, through the following steps.

### Mold Preparation Step

First, as illustrated in FIG. 9(A), a mold 61 is prepared. The mold 61 includes a surface 61a provided with a fine structure 61b. The fine structure 61b includes a plurality of cone structures 61c. The mold 61 can be produced by machining a flat plate including a material such as a metal or a resin by using a diamond cutter or a laser. The cone structure 61c has substantially the same shape as the cone structure 31 of the adhesive sheet 110. A difference in the size between the cone structure 61c and the cone structure 31 is preferably within ±5%, within ±3%, or within ±1%. However, with respect to the height H of the cone structure 31, a greater difference may be caused due to influence of shrinkage of the second part 5 and gravity. Note that the size of the cone structure 31 means the size obtained, for example, within 5 minutes or within 3 minutes immediately after the liner 71 is peeled.

### Liner Production Step

Next, as illustrated in FIGS. 9(A) to 9(C), the mold 61 is pressed against the liner 71, to transfer the fine structure 61b of the surface 61a of the mold 61 to the liner 71. Examples of a material of the liner 71 include a material with which the fine structure 61b can be formed by the transfer and can be retained. The liner 71 in an example includes a sheet 71a including a resin laminated on a surface of a sheet body made from a resin or paper, and a release coating 71b provided in a surface of the sheet 71a. The release coating 71b is made from silicone, for example. The fine structure 61b can be transferred by bringing the mold 61 into contact with the surface (release coating 71b) of the liner 71, and heat pressing the surface of the liner 71. A fine structure 72 complementary to the fine structure 61b of the mold 61 is formed by the transfer in the surface of the liner 71. The fine structure 72 includes a plurality of recesses 72a including cone structures.

### First Part Formation Step

Next, as illustrated in FIGS. 10(A) to 10(E), the first part 4 is formed by applying a solution including an adhesive material to the fine structure 72 of the liner 71 and then curing the solution.

First, as illustrated in FIG. 10(A), a solution 81 including an adhesive material is applied by coating, spraying, or the like to the fine structure 72 formed in the surface of the liner 71.

Next, as illustrated in FIG. 10(B), an excess of the solution 81 is scraped off by, for example, a removal device 82 such as a doctor blade or a squeegee. The removal device 82 moves in a direction A along the surface of the liner 71. Accordingly, the solution 81 is reserved in each of the recesses 72a formed in the surface of the liner 71 as illustrated in FIG. 10(C). In the fine structure 72 formed in the surface of the liner 71, when an interval between the recesses 72a is small, it becomes easy to scrape off the solution 81.

As illustrated in FIG. 10(D), the removal device 82 may scrape off an excess of the solution 81 in a state where an interval GP is formed between the removal device 82 and the surface of the liner 71. In this case, the solution 81 also remains between the recesses 72a. The interval GP may be, for example, 200 μm or more and 400 μm or less.

Next, as illustrated in FIG. 10(E), the solution 81 in the recesses 72a is dried, and a solvent is removed. Thus, the first part 4 is formed in each recess 72a. The first part 4 is disposed in a lowermost part of each recess 72a and is made from a solid adhesive material. After drying, as necessary, the first part 4 may be irradiated with an ultraviolet ray, an electron beam, or the like to cure the adhesive material. In an embodiment, as illustrated in FIG. 10(E), the first part 4 occupies a space from the lowermost part to a middle of the recess 72a, and includes, in an upper part side, a surface substantially parallel to the horizontal plane defined by placement of the liner 71 during drying. Note that in the mold 61 used in producing the liner 71, when the angle θ formed between the side surface and the bottom surface of the cone structure 61c is large, or when the distance between the bottom surfaces of the cone structures 61c is small, the solution 81 including the adhesive material can easily reach the lowermost part of the recess 72a. As a result, formation of the first part 4 is also easy. The solution 81 is a solution formed by dissolving and/or dispersing an acrylic adhesive, a rubber adhesive, or a silicone-based adhesive in an appropriate solvent. The solvent used in the solution 81 may also affect the above-described scraping off of the solution 81. For example, when a solvent such as ethyl acetate, methyl ethyl ketone, and methyl isobutyl ketone is used, as the distance between the bottom surfaces of the cone structures 61c in the mold 61 is smaller (e.g. 50 μm or less), the solution 81 is more easily scraped off.

### Second Part Formation Step

Next, as illustrated in FIG. 11(A), a non-adhesive or weak adhesive material or a precursor of the non-adhesive or weak adhesive material is applied to the liner 71 in which the first part 4 is formed, and thus the second part 5 is formed. In the present embodiment, the adhesive layer 12 including the second part 5 and the base 32 is formed on the liner 71. When any other part is present between the first part 4 and the second part 5, the second part 5 may be formed after the formation of the first part 4 and then the formation of the any other part. The application of the non-adhesive or weak adhesive material can be performed by a variety of methods. For example, the non-adhesive or weak adhesive material molded in advance into a sheet shape or the like is applied to the fine structure 72 of the liner 71, and stands under heat and/or pressure, or at normal temperature and normal pressure for a certain period of time or more. Thus, the non-adhesive or weak adhesive material flows and enters the recesses 72a in the surface of the liner 71, and is joined to the first part 4 located at the lowermost part of the recess 72a. In another example, a precursor of the non-adhesive or weak adhesive material is applied by coating to the fine structure 72 of the liner 71, enters the recesses 72a, and then is irradiated with energy rays. In another example, a solution of the non-adhesive or weak adhesive material is applied by coating to the fine structure 72 of the liner 71, enters the recesses 72a, then heated as necessary, and dried to remove the solvent.

### Carrier Formation Step

Next, as illustrated in FIGS. 11(B) to 11(C), the carrier 102 such as a PVC film is formed on the adhesive layer 12. The carrier 102 is laminated onto the adhesive layer 12, for example, by using a roller 103.

Through the steps described above, the adhesive sheet 110 of FIG. 3 can be manufactured. After the second part 5 is formed as illustrated in FIG. 11(A), the liner 71 may be peeled from the adhesive layer 12 without forming the carrier 102. In this case, the adhesive sheet 10 of FIGS. 1 and 2 can be manufactured. Additionally, when the second part 5 is formed, a pair of the liners 71 (see FIG. 10(E)) in which the first part 4 remains in the recess 72a may be prepared, and a non-adhesive or weak adhesive material or a precursor of the non-adhesive or weak adhesive material may be disposed between the pair of liners 71. In this case, the adhesive sheet 210 of FIG. 4 can be manufactured. Further, the adhesive sheet 310 of FIG. 5 can be manufactured in a manner similar to that described above by changing the shape of the fine structure 61b of the mold 61.

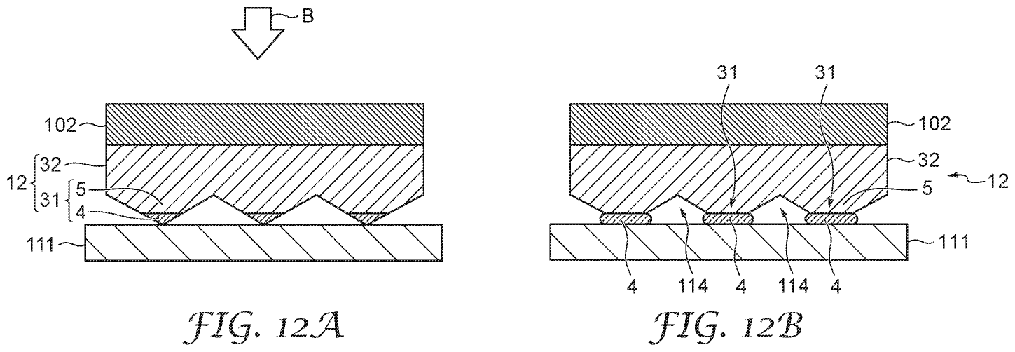

FIG. 12 is a cross-sectional view illustrating a step of applying the adhesive sheet 110 of FIG. 3 to a subject. First, as illustrated in FIG. 12(A), the liner 71 is removed from the adhesive sheet 110, and the adhesive sheet 110 is placed on a subject 111 such that the first part 4 of the adhesive layer 12 faces the subject 111. The subject 111 may be, for example, an object such as a wall material, a floor material, a tile material, a sash material, and a signboard.

Pressure (arrow B) is applied to the carrier 102, and thus the adhesive layer 12 is applied to the subject 111 as illustrated in FIG. 12(B). Accordingly, the first part 4 is bonded to the subject 111, and is also crushed to deform. At this time, the second part 5 almost does not deform, and thus the gap 114 is formed between the cone structures 31 adjacent to each other. The gap 114 is formed between the second parts 5 adjacent to each other. The gap 114 may be formed between the first parts 4 adjacent to each other. After the adhesive layer 12 is applied to the subject 111, the first parts 4 adjacent to each other may come into contact with each other. Similarly, in this case, the gap 114 is formed between the second parts 5 adjacent to each other.

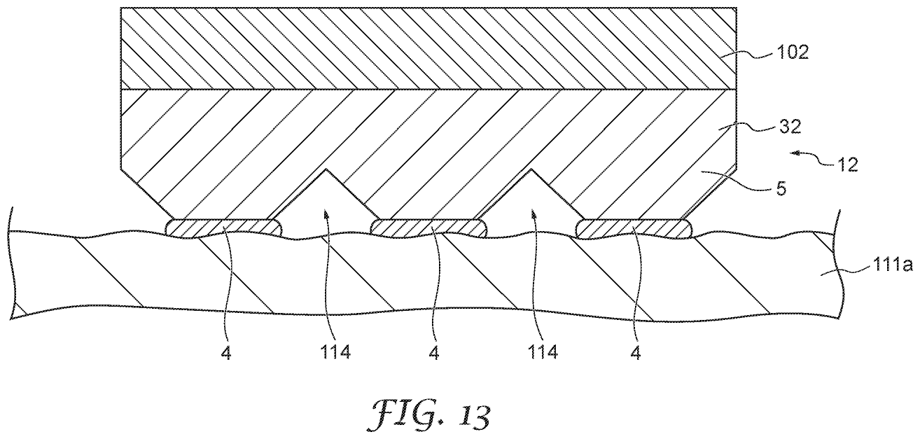

FIG. 13 is a cross-sectional view illustrating an adhesive sheet attached to another subject. As illustrated in FIG. 13, the adhesive layer 12 is applied to a subject 111a by applying pressure to the carrier 102. The subject 111a includes an uneven surface. The subject 111a may be, for example, a superficial part of a human body or an animal such as skin. Even when the subject 111a includes an unevenness surface, after the adhesive layer 12 is applied to the subject 111a, the gap 114 is formed between the cone structures 31 adjacent to each other.

Similarly, the adhesive sheets 10, 210, and 310 can be applied to the subject 111, 111a. In any case, the gap is formed between the convex structures adjacent to each other.

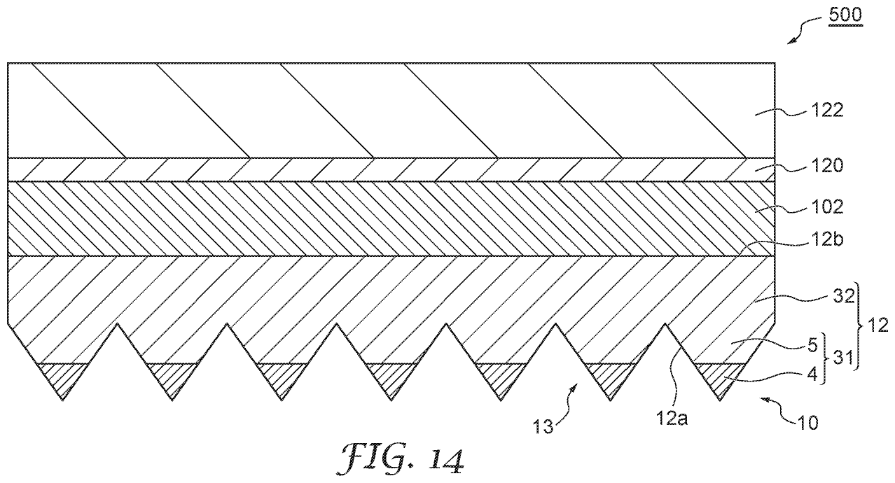

FIG. 14 is a cross-sectional view of a device laminate according to an embodiment. The device laminate 500 illustrated in FIG. 14 includes the adhesive sheet 10 (see FIGS. 1 and 2), the carrier 102 as a substrate, a connection layer 120, and a device 122. The carrier 102 is disposed between the adhesive sheet 10 and the device 122. The connection layer 120 is disposed between the carrier 102 and the device 122. The adhesive layer 12 of the adhesive sheet 10 includes the surface 12b close to the carrier 102 and the surface 12a far from the carrier 102. The adhesive layer 12 includes the surface 12a provided with the fine structure 13.

The device laminate 500 may be a wearable device that is applied, for example, to skin. The device 122 may be, for example, an electrical storage device such as a lithium ion secondary battery, or an electronic device such as a computer. The connection layer 120 may be, for example, an adhesive layer or a mechanical fastener including a hook and a loop.

In the above-described device laminate 500, after the cone structures 31 of the adhesive sheet 10 are applied to the subject 111, 111a, a gap can be formed between the cone structures 31 adjacent to each other. Thus, even when the device 122 has no breathability, after the cone structures 31 of the adhesive sheet 10 are applied to skin, water vapor generated from the skin is released to the outside through the gap. Additionally, adhesiveness between the adhesive sheet 10 and the subject 111, 111a can be reduced by making a stripping solution flow in the gap. Thus, the device laminate 500 can be peeled off from the subject 111, 111a in a short time, and the device laminate 500 and the subject 111, 111a (e.g. skin) are less likely to be damaged.

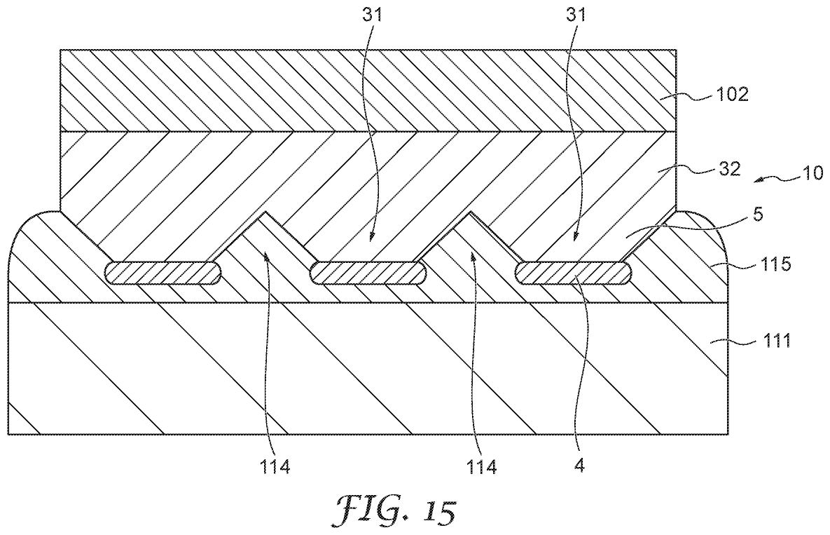

FIG. 15 is a cross-sectional view illustrating a step in a method for peeling an adhesive sheet according to an embodiment. In the method for peeling an adhesive sheet according to the present embodiment, as illustrated in FIG. 15, the adhesive sheet 10 is peeled from the subject 11 by making the stripping solution 115 flow in the gap 114 formed between the cone structures 31 adjacent to each other, in a state where the cone structures 31 of the adhesive sheet 10 are applied to the subject 111. For example, a 3M™ Cavilon™ remover for skin and a Brava brand adhesive remover are used as the stripping solution 115.

In the peeling method according to the present embodiment, adhesiveness between the adhesive sheet 10 and the subject 111 can be reduced by making the stripping solution 115 flow in the gap 114. Thus, the adhesive sheet 10 can be peeled from the subject 111 in a short time, and the adhesive sheet 10 and the subject 111 are less likely to be damaged.

### EXAMPLES

The present disclosure will be described in detail below by way of examples, but the present disclosure is not intended to be limited to the examples.

### Example 1

An adhesive sheet of Example 1 including a structure in which the liner 71 was removed from the adhesive sheet 110 of FIG. 11 was produced as follows.

First, a mold (corresponding to the liner 71) was prepared by applying a polypropylene sheet including a surface provided with a plurality of square pyramids arrayed in a lattice shape, to an SUS plate having a thickness of 1 mm. The height of the square pyramid (corresponding to H of FIG. 5) was 65 μm, and an interval (corresponding to B of FIG. 5) between apices of the square pyramids adjacent to each other was 100 μm.

Next, a solution including the material of the first part 4 was applied by coating onto the mold by using a knife coater to fill gaps formed between the square pyramids. An interval between the mold and the knife coater was 300 μm. A solution including 100 parts by mass of an acrylic polymer solution (isooctyl acrylate:acrylate acid=90:10, solid content 18 mass %), 4.5 parts by mass of a plasticizer (isooctyl palmitate, product name: NIKKOL SG-IOP, manufactured by Nikko Chemicals Co., Ltd.), 0.05 parts by mass of a dye (product name: High orange LH, manufactured by Daiwa Dyestuff Mfg. Co., Ltd.), 50 parts by mass of isopropyl alcohol, and 50 parts by mass of toluene was used as the solution including the material of the first part 4. Solid content of the solution was 10%. After the coating, the solution was dried in an oven at 120° C. to form the first part 4. A storage elastic modulus (G′) at 37° C. of the first part 4 was 1.73×10⁴ Pa.

Next, a solution including the material of the second part 5 was applied by coating onto the mold by using a knife coater to fill the gaps formed between the square pyramids. An interval between the mold and the knife coater was 1 mm. A solution including 100 parts by mass of an acrylic polymer solution (isooctyl acrylate:acrylate acid=90:10, solid content 18 mass %), 3 parts by mass of a crosslinking agent (1′-isophthaloylbis (2-methylaziridine), manufactured by 3M, solid content: 3%), 50 parts by mass of isopropyl alcohol, and 50 parts by mass of toluene was used as the solution including the material of the second part 5. Solid content of the solution was about 9%. After the coating, the solution was dried in an oven at 120° C. to form the second part 5. A storage elastic modulus (G′) at 37° C. of the second part 5 was 1.07×10⁵ Pa. Accordingly, the adhesive layer was formed on the mold (see FIG. 11(A)).

Next, a PET film (thickness 25 μm) as a carrier was laminated on the adhesive layer, and then the mold was removed to produce the adhesive sheet of Example 1.

### Example 2

An adhesive sheet of Example 2 was produced in a similar manner to that in Example 1 except that, in a solution including the material of the first part 4, 18 parts by mass of a plasticizer and 0.5 parts by mass of a dye were used, and an interval between a mold and a knife coater in applying by coating the solution including the material of the first part 4 was 200 μm, and that in a solution including the material of the second part 5, 6 parts by mass of a crosslinking agent was used. A storage elastic modulus (G′) at 37° C. of the first part 4 was 4.05×10⁴ Pa. A storage elastic modulus (G′) at 37° C. of the second part 5 was 1.25×10⁵ Pa.

### Example 3

An adhesive sheet of Example 3 was produced in a similar manner to that in Example 2 except that an interval between a mold and a knife coater in applying by coating a solution including the material of the first part 4 was 300 μm

### Example 4

An adhesive sheet of Example 4 was produced in a similar manner to that in Example 3, except that, in a solution including the material of the second part 5, 12.6 parts by mass of a boron nitride filler (product name: Platelets012, manufactured by 3M) was used instead of the crosslinking agent, and solid content of the solution was 11%, and that a surgical tape (product name: Blenderm (trade name), manufactured by 3M) was used instead of the PET film as a carrier. A storage elastic modulus (G′) at 37° C. of the second part 5 was 4.13×10⁵ Pa.

### Reference Example 1

An adhesive sheet of Reference Example 1 was produced in a similar manner to that in Example 1 except that an interval between a mold and a knife coater in applying by coating a solution including the material of the first part 4 was 1 mm, and that the second part 5 was not formed.

### Reference Example 2

An adhesive sheet of Reference Example 2 was produced in a similar manner to that in Example 1 except that the first part 4 was not formed.

### Reference Example 3

An adhesive sheet of Reference Example 3 was produced in a similar manner to that in Reference Example 1 except that a mold including a flat surface was used instead of the mold including the surface provided with the plurality of square pyramids.

### Reference Example 4

An adhesive sheet of Reference Example 4 was produced in a similar manner to that in Example 3 except that an interval between a mold and a knife coater in applying by coating a solution including the material of the first part 4 was 1 mm, and that the second part 5 was not formed.

### Reference Example 5

An adhesive sheet of Reference Example 5 was produced in a similar manner to that in Example 3 except that the first part 4 was not formed.

### Reference Example 6

An adhesive sheet of Reference Example 6 was produced in a similar manner to that in Reference Example 4 except that a mold including a flat surface was used instead of the mold including the surface provided with the plurality of square pyramids.

### Reference Example 7

An adhesive sheet of Reference Example 7 was produced in a similar manner to that in Reference Example 5 except that a mold including a flat surface was used instead of the mold including the surface provided with the plurality of square pyramids.

### Reference Example 8

An adhesive sheet of Reference Example 8 was produced in a similar manner to that in Example 4 except that the first part 4 was not formed.

### Reference Example 9

An adhesive sheet of Reference Example 9 was produced in a similar manner to that in Example 4 except that an interval between a mold and a knife coater in applying by coating a solution including the material of the first part 4 was 1 mm, and that the second part 5 was not formed.

### Evaluation Results

The adhesive sheets of Examples 1 to 4 and Reference Examples 1 to 9 were evaluated as follows.

### 180° Peel Adhesion Strength

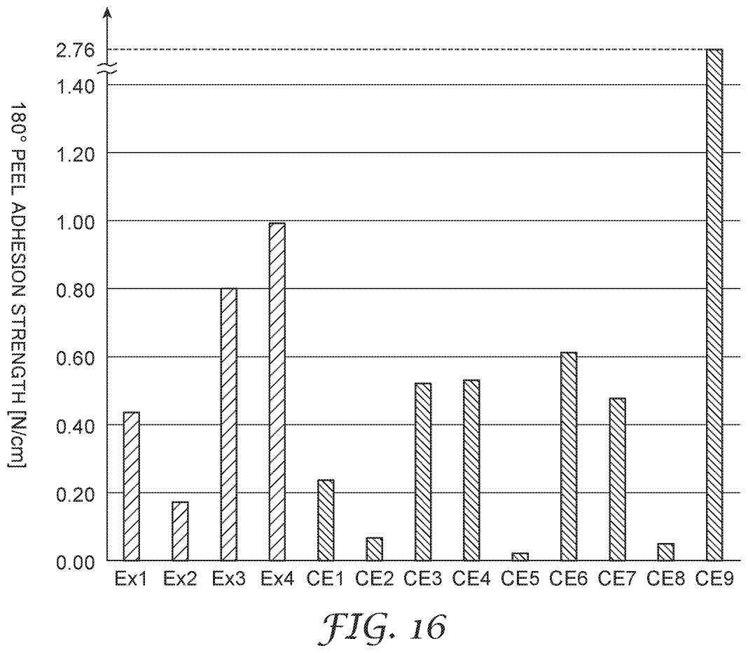

First, each adhesive sheet was cut to have a width of 10 mm, laminated on an SUS304BA plate, and subjected to compression bonding by reciprocating a roller of 2 kg. Immediately after the compression bonding, the adhesive sheet was pulled in the 180° direction at a speed of 300 mm/min by using a peel test device (product name: RTG-1250, manufactured by A&D) to be peeled from the SUS304BA plate. In this manner, 180° peel strength of each adhesive sheet was measured. FIG. 16 shows the results.

FIG. 16 is a graph showing 180° peel adhesion strength of the adhesive sheets of the examples and the reference examples. In FIG. 16, Ex represents an example, and CE represents a reference example. As can be seen in FIG. 16, adhesiveness of the adhesive sheets in Reference Examples 2, 5, and 8 was very low. On the other hand, adhesiveness of the adhesive sheet in Reference Example 9 was very high.

### Appearance

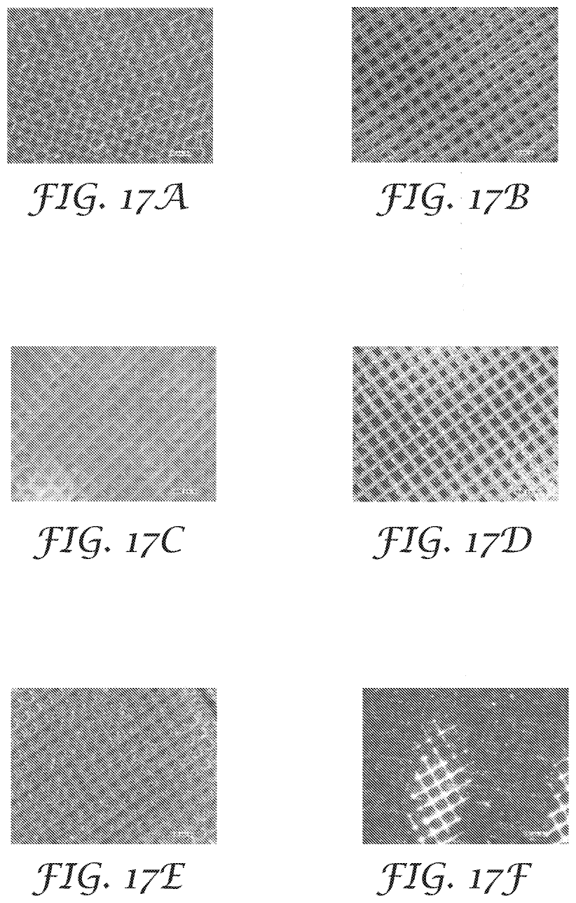

First, each of the adhesive sheets of Examples 1 to 3 and Reference Examples 1 to 5 was laminated on an SUS304BA plate, and subjected to compression bonding by reciprocating a roller of 2 kg. Additionally, each of the adhesive sheets of Example 4 and of Reference Examples 8 and 9 was laminated on a glass plate, and subjected to compression bonding by reciprocating a roller of 2 kg. Appearance of the square pyramid of each of the adhesive sheets was observed from the PET film side by using a microscope (VHX500, manufactured by KEYENCE CORPORATION). FIG. 17 illustrates the results for Examples 1 and 2 and Reference Example 1.

FIG. 17 is a drawing illustrating the adhesive sheets of the examples and the reference examples before and after the application. FIG. 17(A) illustrates the adhesive sheet of Example 1 before the application to the SUS304BA plate. FIG. 17(B) illustrates the adhesive sheet of Example 1 after the application to the SUS304BA plate. FIG. 17(C) illustrates the adhesive sheet of Example 2 before the application to the SUS304BA plate. FIG. 17(D) illustrates the adhesive sheet of Example 2 after the application to the SUS304BA plate. FIG. 17(E) illustrates the adhesive sheet of Reference Example 1 before the application to the SUS304BA plate. FIG. 17(F) illustrates the adhesive sheet of Reference Example 1 after the application to the SUS304BA plate.

As illustrated in FIGS. 17(A) to 17(D), as for Examples 1 and 2, the gap formed between the square pyramids adjacent to each other was retained after the application of the adhesive sheets to the subject. Similarly, as for Example 3, the gap was maintained after the application. On the other hand, as illustrated in FIGS. 17(E) to 17(F), as for Reference Example 1, the gap formed between the square pyramids adjacent to each other was crushed after the application of the adhesive sheet to the subject. Similarly, as for Reference Examples 2 to 5, the gap was not maintained after the application. Note that the adhesive sheets of Reference Examples 3, 6, and 7 include the surface of the adhesive layer provided with no square pyramid.

### Peel Test Using Stripping Solution

Each of the adhesive sheets of Example 3 and Reference Example 3 was cut to have a size of 19 mm×50 mm, laminated on an SUS304BA plate, and subjected to compression bonding by reciprocating a roller of 2 kg. Immediately after the compression bonding, the adhesive sheet was pulled in the 180° direction at a speed of 300 mm/min by using a peel test device (product name: RTG-1250, manufactured by A&D) to be peeled from the SUS304BA plate. In this manner, 180° peel strength of each adhesive sheet was measured. The 180° peel strength of the adhesive sheet of Example 3 was 1.33 N/19 mm. The 180° peel strength of the adhesive sheet of Reference Example 3 was 3.79 N/19 mm.

Next, each of the adhesive sheets of Example 3 and Reference Example 3 was cut to have a size of 19 mm×50 mm, laminated on an SUS304BA plate, and subjected to compression bonding by reciprocating a roller of 2 kg. When 20 minutes elapsed after the compression bonding, a stripping solution (remover for skin, product name: Cavilon™ remover manufactured by 3M Company) was dripped to a vicinity of an interface between the adhesive sheet and the SUS304BA plate. Immediately after the dripping, the adhesive sheet was pulled in the 180° direction at a speed of 300 mm/min by using a peel test device (product name: RTG-1250, manufactured by A&D) to be peeled from the SUS304BA plate. In this manner, 180° peel strength of each adhesive sheet was measured. The 180° peel strength of the adhesive sheet of Example 3 was 0.04 N/19 mm. The 180° peel strength of the adhesive sheet of Reference Example 3 was 3.51 N/19 mm.

As for the adhesive sheet of Example 3, a significant decrease in adhesive force between the adhesive sheet and the SUS304BA plate due to the use of the stripping solution was confirmed. On the other hand, as for the adhesive sheet of Reference Example 3, there was no significant decrease in adhesive force between the adhesive sheet and the SUS304BA plate even when the stripping solution was used. As for the adhesive sheet of Example 3, since the stripping solution passed through the gap formed between the square pyramids adjacent to each other, and spread in all the surface of the adhesive sheet, adhesive force decreased in all the surface of the adhesive sheet. On the other hand, as for the adhesive sheet of Reference Example 3, adhesive force decreased in a part where the stripping solution was dripped, but adhesive force was maintained in other parts.

### REFERENCE SIGNS LIST

1 . . . Bottom surface, 12a, 12b, 112a, 112b . . . Surface, 2 . . . Top, 3 . . . Side surface, 4, 14 . . . First part, 5, 15 . . . Second part, 11, 111, 111a . . . Subject, 12, 112, 212 . . . Adhesive layer, 13, 113 . . . Fine structure, 10, 110, 210, 310, 410 . . . Adhesive sheet, 114 . . . Gap, 115 . . . Stripping solution, 120 . . . Connection layer, 122 . . . Device, 500 . . . Device laminate.

1. An adhesive sheet comprising:
an adhesive layer including a surface provided with a fine structure;
wherein the fine structure comprises:
a plurality of convex structures;
each of the plurality of convex structures includes two or more parts joined to each other via an interface;
the two or more parts include a first part present at a top of the convex structure and made from an adhesive material, and a second part present in a lower side of the first part, and the second part is harder than the first part.

2. The adhesive sheet according to claim 1, wherein the second part has a storage elastic modulus higher than a storage elastic modulus of the first part.

3. The adhesive sheet according to claim 1, wherein a storage elastic modulus of the second part is 1×10⁵ Pa or more.

4. The adhesive sheet according to claim 1, wherein a storage elastic modulus of the first part is less than 1×10⁵ Pa.

5. The adhesive sheet according to claim 1, wherein an angle θ formed between a side surface and a bottom surface of the convex structure is 30° or more.

6. The adhesive sheet according to claim 1, wherein when a height of the convex structure is 100%, a height of the first part is in the range of 10% to 90% of the height of the convex structure.

7. The adhesive sheet according to claim 1, wherein an interval between bottom surfaces of adjacent ones of the plurality of convex structures in an array direction of the plurality of convex structures is 500 μm or less.

8. A device laminate comprising:
the adhesive sheet according to claim 1;
a device;
a substrate disposed between the adhesive sheet and the device; and
a connection layer disposed between the substrate and the device,
wherein the adhesive layer of the adhesive sheet includes a surface close to the substrate and a surface far from the substrate, and the adhesive layer includes the fine structure in the surface far from the substrate.

9. A method for peeling an adhesive sheet, the method comprising a step of peeling the adhesive sheet according to claim 1 from a subject in a state where the plurality of convex structures of the adhesive sheet are applied to the subject, by making a stripping solution flow in a gap formed between adjacent ones of the plurality of convex structures.
